(19) United States
(12) Patent Application Publication
(10) Pub. No.: US 2020/0281573 A1
(43) Pub. Date: Sep. 10, 2020

(54) Devices for Collecting Human Semen

(71) Applicant: Dmitri Dozortsev, Houston, TX (US)

(72) Inventor: Dmitri Dozortsev, Houston, TX (US)

(21) Appl. No.: 16/429,638

(22) Filed: Jun. 3, 2019

Related U.S. Application Data
(60) Provisional application No. 62/815,576, filed on Mar. 8, 2019.

(51) Int. Cl. A61B 10/00

(52) U.S. Cl. CPC A61B 10/0058

(57) ABSTRACT

Provided herein are collection devices for semen. The collection device may be a collection cup with an open top and a closed bottom with a collection cavity therewithin. The open top and closed bottom are oriented such that an angle θ greater than 0 degrees is formed by an intersection of a plane across the open top with a plane across the closed bottom. Alternatively, the collection cup is removably attached to a flow-through receptacle which is in fluid communication therewith.

### CROSS-REFERENCE TO RELATED APPLICATION

This non-provisional application claims benefit of priority under 35 U.S.C. § 119(e) of provisional application U.S. Ser. No. 62/815,576, filed Mar. 8, 2019, the entirety of which is hereby incorporated by reference.

### BACKGROUND OF THE INVENTION

### Field of the Invention

The invention relates to the field of human infertility. More specifically, the present invention is directed to devices for collecting a sperm sample for an assisted conception procedure or an analysis.

### Description of the Related Art

Assisted fertilization techniques require human sperm. Typically, the sperm is produced by masturbation followed by an ejaculation into a specimen collection cup, routinely, a container with even sized walls. The container is closed with a lid that is oriented perpendicular to the vertical axis of the container, parallel to the bottom of the container.

For collection, the open end of the collection cup is held in front of the glans of the erect penis, close enough to the urethra, to receive an ejaculate before it splatters. Current sperm collection methods have several problems. If the collection cup is positioned too close to the urethra, it makes masturbation difficult. Conversely, if the collection cup is positioned too far, a portion of the ejaculate may get lost due to the splatter and turbulence. Additionally, since the penis is erect the collection cup would need to be held in bottom-up (partially inverted) position, which results in sample loss due to ejaculate leaking to the floor. Furthermore, since these methods require both hands of the donor to be occupied for collection, it becomes difficult to turn a page in a magazine or change a video feed, which may be necessary for achieving optimal orgasm and thus optimal ejaculate volume. The time required to achieve an orgasm may also increase leading to increased collection time.

Thus, there is a deficiency in the art for devices and methods that yield optimal semen collection in the shortest possible time. The present invention fulfills this longstanding need and desire in the art.

### SUMMARY OF THE INVENTION

The present invention is directed to a collection cup for collecting a semen sample. The collection cup comprises an open top portion and a closed bottom portion whereby the portions form a collection cavity therewithin. The closed bottom portion of the collection cup is in an orientation with the open top portion such that an angle θ greater than 0 degrees is formed by an intersection of a plane across the open top with a plane across the closed bottom.

The present invention is directed to a related collection cup having the features described above and further comprising a cap in a removable covering relationship with the open top portion of the collection cup. The present invention is directed to another related collection cup having the features described above and further comprising a hands-free holder releasably gripping the bottom portion of the collection cup where the hands-free holder comprises an adjustable arm with a grip at a proximal end thereof and a base at a distal end thereof.

The present invention also is directed to a semen collection device. The device comprises a collection cup and a flow-through receptacle. The collection cup has an open top and a closed bottom and forms a collection cavity therewithin. The flow-through receptacle comprises a proximal open end and a distal open end removably engaged with the open top of the collection cup and in fluid communication with the collection cavity.

The present invention is directed to a related semen collection device having the features described above and further comprising a cap in a removable covering relationship with the distal open end of the flow-through receptacle. The present invention is directed to another related a semen collection device having the features described above and further comprising a hands-free holder releasably gripping the collection cup where the hands-free holder comprising an adjustable arm with a grip at a proximal end thereof and a base at a distal end thereof.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1D show the various elements of the collection cup. FIG. 1A is a side view of the collection cup in a vertical orientation showing a dome-shaped open top and a closed bottom. FIG. 1B is a front view of the collection cup shown in FIG. 1A showing the dome-shaped open top and a closed bottom. FIG. 1C is cross sectional view of the collection cup shown in FIG. 1B, showing the open top and the tapered closed bottom. FIG. 1D is an interior view of the collection cup shown in FIG. 1B showing the collection cavity formed within the collection cup between the tapered closed bottom and the open top.

FIGS. 2A-2B show a semen collection device, an alternate configuration of the collection cup. FIG. 2A is a side view of the device in a vertical orientation showing a collection cup that is attached at the open top to a proximal end of a dome-shaped flow-through receptacle for receiving an ejaculate.

FIGS. 3A-3B show an alternate configuration of the flow-through receptacle on the semen collection device. FIG. 3A is a side view of the device in a vertical orientation showing a substantially L-shaped hemicylindrical flow-through receptacle for receiving an ejaculate. FIG. 3B is a front view of the device shown in FIG. 3A.

FIG. 4 shows a hands-free holder and illustrates the simultaneous hands-free gripping of the collection cup and attachment to a chair armrest for the semen collection.

### DETAILED DESCRIPTION OF THE PRESENT INVENTION

For convenience, before further description of the present invention, certain terms employed in the specification, examples and appended claims are collected herein. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

The term “or” in the claims refers to “and/or” unless explicitly indicated to refer to alternatives only or the alternatives are mutually exclusive, although the disclosure supports a definition that refers to only alternatives and “and/or”.

The terms “comprise” and “comprising” are used in the inclusive, open sense, meaning that additional elements may be included.

The term “including” is used herein to mean “including, but not limited to”. “Including” and “including but not limited to” are used interchangeably.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure. For example, the angle θ as disclosed herein may be greater than 0 degrees to about 90 degrees which encompasses 100 degrees at the upper end.

As used herein, the terms “collection cup”, “cup”, “collection device”, “semen collection device”, and “device” are interchangeable as are “open top end” of the collection cup and “flow-through receptacle” of the semen collection device.

As used herein, the terms “proximal” refers to nearer or nearest to the collection cup and “distal” refers to farther or farthest from the collection cup.

In one embodiment of the present invention, there is provided collection cup for collecting a semen sample comprising an open top portion; and a closed bottom portion in an orientation with the open top portion such that an angle θ greater than 0 degrees is formed by an intersection of a plane across the open top with a plane across the closed bottom, said open top portion and closed bottom portion forming a collection cavity therewithin.

Further to this embodiment the collection cup comprises a cap in a removable covering relationship with the open top portion of the collection cup. In an aspect of this further embodiment the open top portion may comprise threads exteriorly and circumferentially disposed thereon and the cap may comprise threads interiorly and circumferentially disposed therein where the cap is threadably engageable with the open top portion of the collection cup.

In another further embodiment the collection cup comprises a hands-free holder releasably gripping the collection cup where the hands-free holder comprising an adjustable arm with a grip at a proximal end thereof and a base at a distal end thereof. In this further embodiment the base may be attachable to a stationary surface.

In all embodiments and aspects thereof the angle θ that the plane across the open top portion end forms with the plane across the bottom portion may be about 1 degree to about 90 degrees. Also in all embodiments and aspects the open top portion may be substantially dome-shaped. In addition the closed bottom portion may be substantially cylindrical. Furthermore the collection cavity may taper toward the bottom portion. Further still the collection cup may be made from at least one biocompatible material selected from the group consisting of a plastic, a plastic composite, a metal and a metal composite.

In another embodiment of the present invention there is provided a semen collection device comprising a collection cup with an open top and a closed bottom forming a collection cavity therewithin; and a flow-through receptacle comprising a proximal open end and a distal open end removably engaged with the open top of the collection cup and in fluid communication with the collection cavity.

Further to this embodiment the semen collection device comprises a cap in a removable covering relationship with the distal open end of the flow-through receptacle. In an aspect of this further embodiment the flow-through receptacle may comprise threads exteriorly and circumferentially disposed at the distal open end and the cap may comprise threads interiorly and circumferentially disposed therein where the cap is threadably engageable with the receptacle at the distal open end.

In another further embodiment the collection cup comprises a hands-free holder releasably gripping the collection cup where the hands-free holder comprises an adjustable arm with a grip at a proximal end thereof and a base at a distal end thereof. In this further embodiment the base may be attachable to a stationary surface.

In all embodiments and aspects thereof the closed bottom of the collection cup may be oriented with the distal open end of the flow-through receptacle such that an angle θ of about 1 degree to about 90 degrees is formed by an intersection of a plane across the closed bottom with a plane across the distal open end. Also in all embodiments and aspects the the flow-through receptacle may be substantially dome-shaped or may be flow-through receptacle is substantially dome-shaped or is substantially hemicylindrical. In addition the collection cup may be substantially cylindrical. Furthermore the collection cavity may taper toward the closed bottom of the collection cup. Further still the flow-through receptacle and the collection cup may be made from at least one biocompatible material selected from the group consisting of a plastic, a plastic composite, a metal and a metal composite.

Provided herein is a collection cup or collection device comprising a collection cup for collecting human semen. Alternatively, the collection cup or collection device may be used for collecting a urine sample. The collection cup may be a single piece or the semen collection device may comprise two pieces, for example, a flow-through receptacle and a collection cup that are removably engageable or attachable and when engaged are in fluid communication. Engaging, attaching or securing the flow-through receptacle to the collection cup may be accomplished using any means that enables quick disengagement of the receptacle from the collection cup to collect the semen. For example, the flow through receptacle and the collection cup may comprise threads and are thus threadably engageable. Alternatively, the receptacle may removably snap onto the collection cup. The collection cup or collection device may be fitted with a removable cap that is threadably engageable with a suitably threaded collection cup or flow-through receptacle or, alternatively, may snap thereon. The cap enables storage of the collected sample.

The collection cup and collection device and cap may be made from any suitable biocompatible material or combination of biocompatible materials. Biocompatible materials include, but are not limited to, plastic, metal, a plastic composite, a metal composite or a combination of these materials.

The collection cup and the flow-through receptacle are configured to have any suitable shape and size to receive a semen ejaculate or specimen from an erect penis or, alternatively, a urine specimen, into the collection cavity and to enable an easy retrieval of the specimens with a laboratory pipette from any area in the bottom portion of the collection cup to retrieve the semen. For example, the open top portion of the single piece collection cup may be substantially dome-shaped and the closed bottom portion may be substantially cylindrical. In the two-piece semen collection device the collection cup may have any suitable shape and size to complement the shape and size of the receptacle. For example, the flow-through receptacle may be substantially dome-shaped, may be a substantially hemicylindrical or may be a rectangular cuboid and the collection cup may have a substantially cylindrical or a substantially conical shape.

These shapes are beneficial since it allows the user to comfortably position the open top portion of the collection cup or the distal open end of the collection device in front of the erect penis while holding the cup in upright position without having to tilt the collection cup to a bottom-up position. Additionally, this feature also allows a laboratory pipette, for example, a straight pipette or a flexible pipette, to be inserted into the collection cup to reach any area in the bottom portion to retrieve the semen without having to tilt the collection cup, which is advantageous. Moreover, the orientation of planes across the open top portion and the closed bottom in the collection cup and of the planes across the distal open end of the flow-through receptacle and the closed bottom of the collection cup in the collection device intersect at an angle greater than 0 degrees, for example, about 1 degree to about 90 degrees.

The collection cavity enclosed within the collection cup in either the single-piece cup or two-piece collection device may be either uniformly parallel or progressively tapering to the bottom of the cup. Tapering is beneficial since it prevents loss of semen sample due to splattering and turbulence while keeping the open top portion of the collection cup or the distal open end of the receptacle sufficiently large for receiving an ejaculate. Tapering also is beneficial since it reduces the surface area of the semen deposit in contact with ambient air, thereby reducing evaporation. For example, the tapered collection cavity has a diameter across the open top portion of the collection cup or across the distal open end of the receptacle that is about three times to about twenty times larger than the diameter at the closed bottom of the collection cup or across the proximal open end of the receptacle.

The collection cup or the semen collection device for collecting a semen sample may include a hands-free holder. The holder may be an adjustable arm with a grip at its proximal end and a base at its distal end. The grip releasably grips the collection cup. The adjustable arm may be a flexible arm, a telescopic arm or an arm that swivels from the base. This provides the user with the convenience of maneuvering the arm to position the collection cup or receptacle in line with an erect penis allowing hands-free use of the cup or device. The base is attachable to a stationary surface including, but not limited to, a floor, a chair, a sofa, or a desk. The hands-free holder may be made of any suitable material including, but not limited to, plastic, metal, plastic composites, metal composites or a combination of these materials.

Particularly, embodiments of the present invention are better illustrated with reference to the Figure(s), however, such reference is not meant to limit the present invention in any fashion. The embodiments and variations described in detail herein are to be interpreted by the appended claims and equivalents thereof.

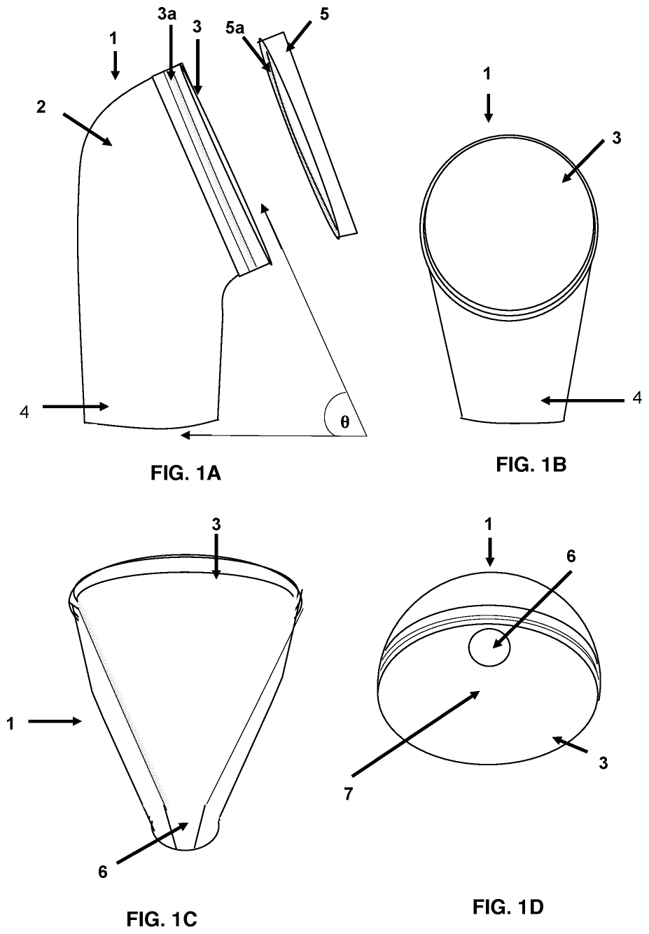

FIG. 1A is a side view of the single-piece semen collection cup 1 showing a dome-shaped open top or open top portion 2 with opening 3 and a closed bottom or closed bottom portion 4. The open top portion has threads 3a exteriorly and circumferentially disposed around the outer surface thereof, which enables a user to optionally close the collection cup with cap 5 with threads 5a interiorly and circumferentially disposed around an inner edge thereof. Angle θ is formed by the intersection of the plane across the open top 2 at the opening 3 with the plane across the closed bottom and generally is greater than 0° and ranging to about 90°.

With continued reference to FIG. 1A, FIG. 1B is a front view of the semen collection cup showing the opening 3 and the closed bottom portion 4.

With continued reference to FIG. 1B, FIG. 1C is a cross sectional view of the semen collection cup showing the opening 3 and the tapering toward the closed bottom 6.

With continued reference to FIGS. 1B and 1C, FIG. 1D is an interior view of the semen collection cup showing the collection cavity 7 formed between the the open top 2 and the tapered closed bottom 6.

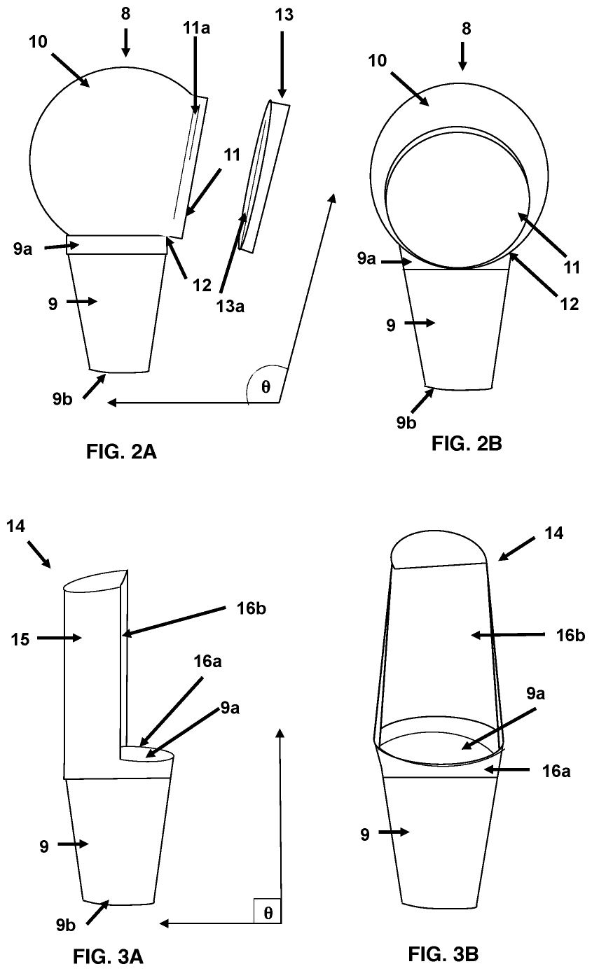

FIG. 2A is a side view of the two-piece semen collection device 8 showing a collection cup 9 engaged with a dome-shaped flow-through receptacle 10. The collection cup 9 has an open top at 9a and a closed bottom at 9b. The dome-shaped flow through receptacle 10 has a distal open end 11 for receiving an ejaculate and a proximal open end 12 shown engaged or attached in fluid communication with the open top 9a on the collection cup. The distal open end 11 has threads 11a exteriorly and circumferentially disposed around the outer surface thereof, which enables a user to optionally close the flow-through receptacle with cap 13 with threads 13a interiorly and circumferentially disposed around an inner edge thereof. Angle θ is corresponds to the angle in FIGS. 1A and 1s formed by the intersection of the plane across the distal open end 11 with the plane across the closed bottom 9b on the collection cup and generally is greater than 0° and ranging to about 90°.

With continued reference to FIG. 2A, FIG. 2B is a front view of the semen collection device 8 showing the distal open end 11 of the dome-shaped flow-through receptacle 10 angled with respect to the closed bottom 9b of the collection cup 9.

FIG. 3A is a side view of an alternate two-piece semen collection device 14 showing the substantially hemicylindrical flow-through receptacle 15 engaging the collection cup 9 and in fluid connection therewith. The hemicylindrical flow-through receptacle 15 is substantially L-shaped in profile with a proximal open end 16a and a distal open end 16b for receiving the semen sample. The proximal open end 16a removably engages the collection cup 9 at the open end 9a. Angle θ is corresponds to the angle in FIG. 2A and is formed by the intersection of the plane across the distal open end 16b with the plane across the closed bottom 9b on the collection cup and generally is about 90°.

With continued reference to FIG. 3A, FIG. 3B is a front perspective view of the semen collection device 14 showing the proximal open end 16a fluidly connecting with the distal open end 16b on the flow-through receptacle 15 and the open end 9a on the collection cup 9.

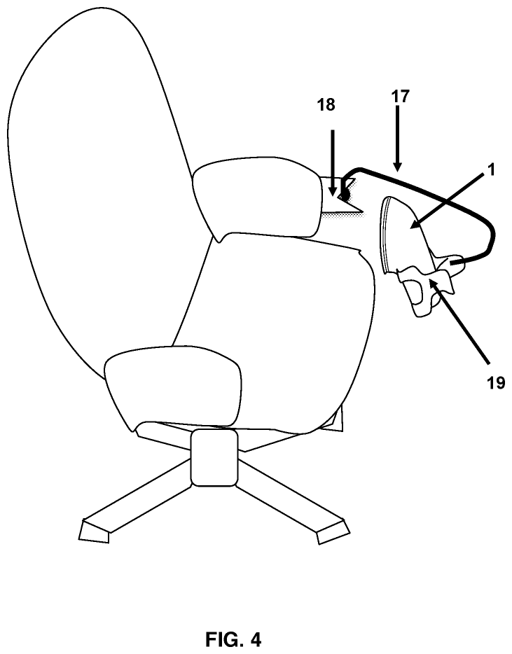

With continued reference to FIGS. 1A-1D, FIG. 4 shows a hands-free holder for the collection cup 1. The hands-free holder is an adjustable arm 17 on which is attached a base 18 at one end and a grip 19 at the other end. The base is secured to a chair armrest. The grip removably secures the semen collection cup 1.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below.

What is claimed is:

1. A collection cup for collecting a semen sample comprising:
an open top portion; and
a closed bottom portion in an orientation with the open top portion such that an angle θ greater than 0 degrees is formed by an intersection of a plane across the open top with a plane across the closed bottom, said open top portion and closed bottom portion forming a collection cavity therewithin.

2. The collection cup of claim 1, further comprising a cap in a removable covering relationship with the open top portion of the collection cup.

3. The collection cup of claim 2, wherein the open top portion comprises threads exteriorly and circumferentially disposed thereon and the cap comprises threads interiorly and circumferentially disposed therein, said cap threadably engageable with said open top portion of the collection cup.

4. The collection cup of claim 1, further comprising a hands-free holder releasably gripping the bottom portion of the collection cup, said hands-free holder comprising an adjustable arm with a grip at a proximal end thereof and a base at a distal end thereof.

5. The collection cup of claim 4, wherein the base is attachable to a stationary surface.

6. The collection cup of claim 1, wherein the angle θ that the plane across the open top portion end forms with the plane across the bottom portion is about 1 degree to about 90 degrees.

7. The collection cup of claim 1, wherein the open top portion is substantially dome-shaped.

8. The collection cup of claim 1, wherein the closed bottom portion is substantially cylindrical.

9. The collection cup of claim 1, wherein the collection cavity tapers toward the bottom portion.

10. The collection cup of claim 1, wherein the collection cup is made from at least one biocompatible material selected from the group consisting of a plastic, a plastic composite, a metal and a metal composite.

11. A semen collection device comprising:
a collection cup with an open top and a closed bottom forming a collection cavity therewithin; and
a flow-through receptacle comprising a proximal open end and a distal open end removably engaged with the open top of the collection cup and in fluid communication with the collection cavity.

12. The semen collection device of claim 11, further comprising a cap in a removable covering relationship with the distal open end of the flow-through receptacle.

13. The semen collection device of claim 12, wherein the flow-through receptacle comprises threads exteriorly and circumferentially disposed at the distal open end and the cap comprises threads interiorly and circumferentially disposed therein, said cap threadably engageable with the receptacle at the distal open end.

14. The semen collection device of claim 11, further comprising a hands-free holder releasably gripping the collection cup, said hands-free holder comprising an adjustable arm with a grip at a proximal end thereof and a base at a distal end thereof.

15. The semen collection device of claim 13, wherein the base is attachable to a stationary surface.

16. The semen collection device of claim 11, wherein the closed bottom of the collection cup is oriented with the distal open end of the flow-through receptacle such that an angle θ of about 1 degree to about 90 degrees is formed by an intersection of a plane across the closed bottom with a plane across the distal open end.

17. The semen collection device of claim 11, wherein the flow-through receptacle is substantially dome-shaped or is substantially hemicylindrical.

18. The semen collection device of claim 11, wherein the collection cup is substantially cylindrical.

19. The semen collection device of claim 11, wherein the collection cavity tapers toward the closed bottom of the collection cup.

20. The semen collection device of claim 11, wherein the flow-through receptacle and the collection cup are made from at least one biocompatible material selected from the group consisting of a plastic, a plastic composite, a metal and a metal composite.
